(19) United States
(12) Patent Application Publication
(10) Pub. No.: US 2006/0116577 A1
(43) Pub. Date: Jun. 1, 2006

(54) Direct image measurement editing mode for ultrasound reports

(75) Inventor: David R. DeWitt, Livermore, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/000,548

(22) Filed: Nov. 30, 2004

(51) Int. Cl. A61B 8/00

(52) U.S. Cl. 600/437

(57) ABSTRACT

A direct image edit mode (DIMEM) introduces flexibility into the ultrasound exam process and, as a result, it improves the workflow associated with it. This flexibility allows a sonographer to seamlessly review, in any order, the entire history of exams. Moreover, with the DIMEM feature images associated with measured values are recalled along with the tools used for measuring the values where the tools are restored to their original state at time of measurement. This allows further review and adjustment of the measured values.

### BACKGROUND

Ultrasound or pulse-echo sonography is a diagnostic imaging technique which uses high-frequency acoustic energy to examine objects and create their visual image on a screen. Ultrasound scans are made by flooding a target area with constant, high-frequency acoustic pulses (ultrasound waves) and capturing reflections of these pulses as they bounce from any object or region boundary encountered in that area. The captured reflections of the pulses (echo waves) are processed electronically and translated into a visual image that provides the contours of such object or region. In medical applications, the ultrasound waves reflect from boundaries between organs and surrounding fluid, and between regions of different tissue density. For example, ultrasound is used to obtain obstetrical scans of fetuses prior to birth and to observe tumors, muscles and bones.

Doppler ultrasound is ultrasound developed to detect movement from phase shifts contained in the echo waves. For example, Doppler ultrasound scans detect moving fluid such a circulating blood which, in turn, can be used to interpret the heartbeat of fetuses.

An ultrasound exam may involve one or more scans where each scan produces an image and from each image one or more measurement values can be derived. Measurement values are derived from a freeze frame of a scan by manipulating 2-dimentional distance calipers, Doppler trace calipers, ellipse or other appropriate measurement tools. For instance, after delimiting its structure the length of an object is obtained by manipulating the distance caliper. This is done by adjusting the distance calipers to set two reference points at each end of the structure and calculating the length between the two reference points to produce the measurement value. Likewise, to measure the contour, circumference or diameter of an object, the ellipse tool is manipulated to encircle the object and calculate the measured value.

In ultrasound exams, workflow is the sequence of steps to produce the scan results, worksheets and reports. Assessment pages such as worksheets and reports are derived from the scan results. Reports contain information from the worksheets in a suitably stylized form. Note that, although the description herein addresses the distinction between worksheets and reports, and this distinction is maintained throughout, for convenience only, the title addresses both of them collectively as reports.

A key feature of diagnostic imaging in present ultrasound systems is the ability to manage digital image data and create structured assessment pages and reports automatically and seamlessly. Scan results, containing digital image data and any measurement values and calculation results, populate assessment pages and final patient reports at the touch of a button, thus eliminating hand-written worksheets and reports. For instance, structured worksheets and reports from ultrasound scans allow physicians and sonographers to meet their clinical and workflow needs for diagnosing and treating patients. Moreover, scan results can flow across a network and, in some instances, scan results can be automatically saved in memory, locally or remotely, with correlation between the digital images and the measurement values. In one implementation, this correlation is provided by automatically incorporating into the digital image data to be saved a measurement-to-image association, namely, a descriptive text of measurement values.

Once scan results are saved they can be retrieved and reviewed. Beyond the ability to review scan results for a completed exam, however, present ultrasound systems are not capable of recreating exam states. Once an exam is completed the state of the tools, for example, is not preserved and there is no way to recall this state for manipulating, verifying or refining any of the results. The only way to accomplish this is to repeat the exam in the same way. Therefore, there is a need for improving workflow which is not being met by the present state of the art.

### SUMMARY

The present invention addresses the foregoing need by providing a direct image measurement editing mode (DIMEM) for ultrasound worksheets and reports. With this approach, workflow improvement in report editing is achieved, among other things, through links between measured values, their respective images and the states of the appropriate measurement tools. As a result, whenever further information or adjustment of measured values is desired the original image can be recalled along with the appropriate tools and their original state at the point when the measured value was produced.

Hence, in accordance with the purpose of the present invention as shown and broadly described herein, one example of an ultrasound system with direct image editing mode includes a worksheet-report generator, one or more measurement tools, a storage and a DIMEM module. Specifically, the generator has an input and an output. The input is for receiving ultrasound scan results of an exam which include an image and one or more instances of a measurement value associated with the image. The output is for providing a worksheet populated with instances of the ultrasound scan results. The measurement tools are set for taking a measurement, wherein each instance of a measurement value is obtained with such a measurement tool. Then, the DIMEM module is operatively connected to the worksheet-report generator and storage. The DIMEM module is configured to preserve in the storage each instance of each measurement value along with information on its associated tool and its setting. Thus, when the image associated with such instance is recalled for further review, the measurement tool used for obtaining such instance is restorable to its setting at the time of taking the measurement. This allows further review and adjustment of such instance.

In further accordance with the purpose of the present invention, a method is provided for improving ultrasound workflow with direct image measurement editing mode. In general terms, one such method includes enabling the direct image measurement editing mode (DIMEM) in an ultrasound system. Then ultrasound scan results are obtained for an exam. These include an image and one or more instances of a measurement value associated with the image, wherein each instance of a measurement value is taken by a measurement tool with settings for taking the measurement. Notably, the ultrasound scan results are then saved along with information about the measurement tool and its original settings at time of measurement. Next a worksheet populated with the instances is provided, allowing navigation to one of the instances which is of interest. When the DIMEM is then invoked, the image associated with this instance of interest is recalled and along with it the tool associated with the instance is restored to its original settings at time of taking the measurement. With the recalled image and restored tools, it is possible to review the instance of interest, adjust the instance of interest, or both. A new worksheet is then generated with the instance of interest, whereby the recall of the image along with the restoration of the associated tool to its settings at time of measurement improves the workflow.

These and other features, aspects and advantages of the present invention will become better understood from the description herein, appended claims, and accompanying drawings as hereafter described.

### BRIEF DESCRIPTION OF THE DRAWINGS

The accompanying drawings which are incorporated in and constitute a part of this specification illustrate various aspects of the invention and together with the description, serve to explain its principles. Wherever convenient, the same reference numbers will be used throughout the drawings to refer to the same or like elements.

FIG. 1 illustrates an ultrasound workflow with direct image measurement edit mode (DIMEM).

FIG. 2 illustrates an ultrasound system in which DIMEM is implemented.

FIG. 3 is a diagram of the relationship between functional components of an ultrasound system with the DIMEM feature.

FIG. 4A illustrates a pre-exam setup screen with selection items including DIMEM and ultrasound scan choices.

FIG. 4B illustrates the DIMEM setup screen.

FIG. 5 is an exemplary freeze frame of an ultrasound scan with the two reference points of a distance caliper set for femur length measurement.

FIG. 6 illustrates the data, such as image, tool, tool state information and exam identification, to be saved along with each of the measured values.

FIG. 7 is an exemplary structured worksheet with tabulated measurement values.

FIG. 8 is an exemplary structured report with data derived from a worksheet.

FIG. 9 shows the exemplary freeze frame from FIG. 5 recalled by the DIMEM feature.

FIG. 10 is a flow diagram of an ultrasound exam workflow with DIMEM.

### DETAILED DESCRIPTION

The present invention is premised on the observation that the workflow in ultrasound exams can be improved and that such improvement can be achieved with the addition of a direct image measurement edit mode (DIMEM). As noted, the salient issue in ultrasound exams is the ability to recall freeze frames of ultrasound scans and verify, edit or adjust the measured values of delimited structures. The addition of DIMEM capability addresses this and related issues.

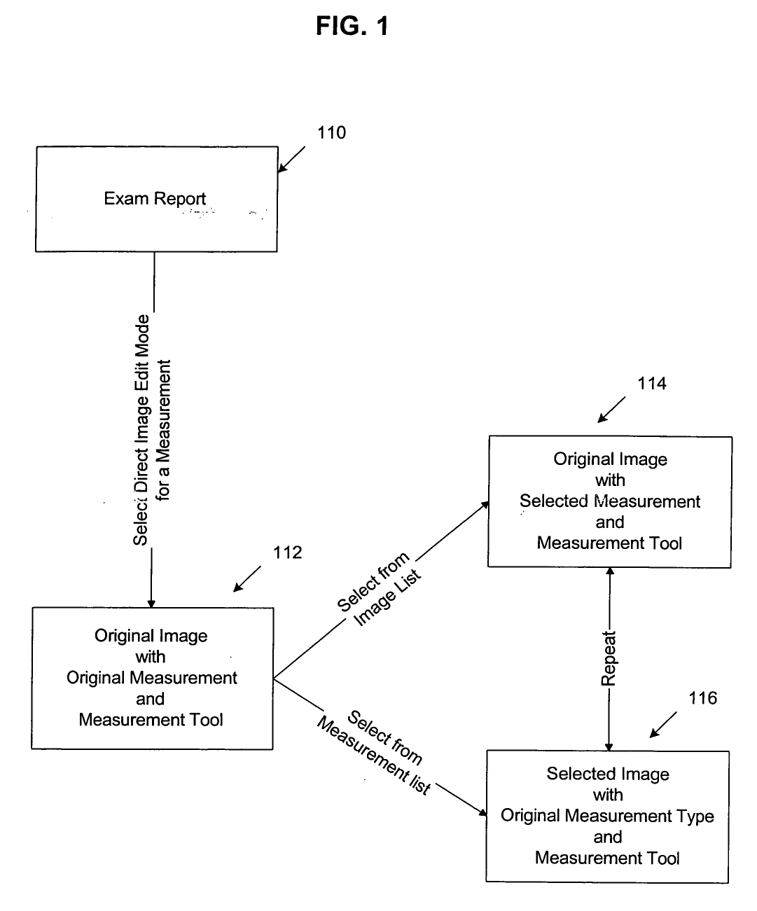

The DIMEM is an added feature that allows review, editing and manipulation of measured values tabulated in the structured worksheet. A first review of workflow with the DIMEM feature is provided in FIG. 1. Starting from the exam worksheet 110, the sonographer invokes the DIMEM from a measured value (instance) that the sonographer touches or navigates a cursor to on the structured worksheet. The invocation of DIMEM enables the sonographer not only to text edit this measure value, say from 6.06 cm to 6.26 cm, but also to recall the freeze frame with the original image of the measured object and to reactivate the measurement tool 112. The measurement tool is reactivated with the same state that it had at the time of the original measurement, and to this end the measurement tool elements are restored to this state (e.g., distance caliper reference points are restored to their position at time of measurement). This allows the sonographer to then adjust or refine this measured value by manipulating the measurement tool away from this state.

In addition to restoration of the image and measurement tool, the DIMEM feature provides additional selections and links capability. In one embodiment this capability is provided with a number of menus (lists) 114 & 116. The measurement list 116 includes a list of all instances of a particular type of measurement taken with images in all the ultrasound exams where DIMEM was enabled for the current patient. The results of a series of exams, such as obstetrical scans in the first, second and third trimesters, produce a historical records that with DIMEM can be seamlessly accessed at the touch of a button (e.g., all femur length measurements from all ultrasound exams for a particular patient). The second menu is an image list 114 that includes a list of all measurements taken with a particular image, namely, the restored image (e.g., all instances of femur length and abdominal circumference measurements taken with the that image for that patient). Then, because DIMEM menus (lists) are recursive where each image is a starting point, the sonographer can start with measurement list 116 and go from there to the image list 114 and vice-versa.

Importantly, in all cases when an image is restored the measurement tool is also restored, and the state to which it is restored is that in which the measurement was taken. Moreover, although the restored image is frozen, representing the freeze frame at the time the measurement is taken, the measurement value can be adjusted because the measurement tool is active and its elements can be manipulated.

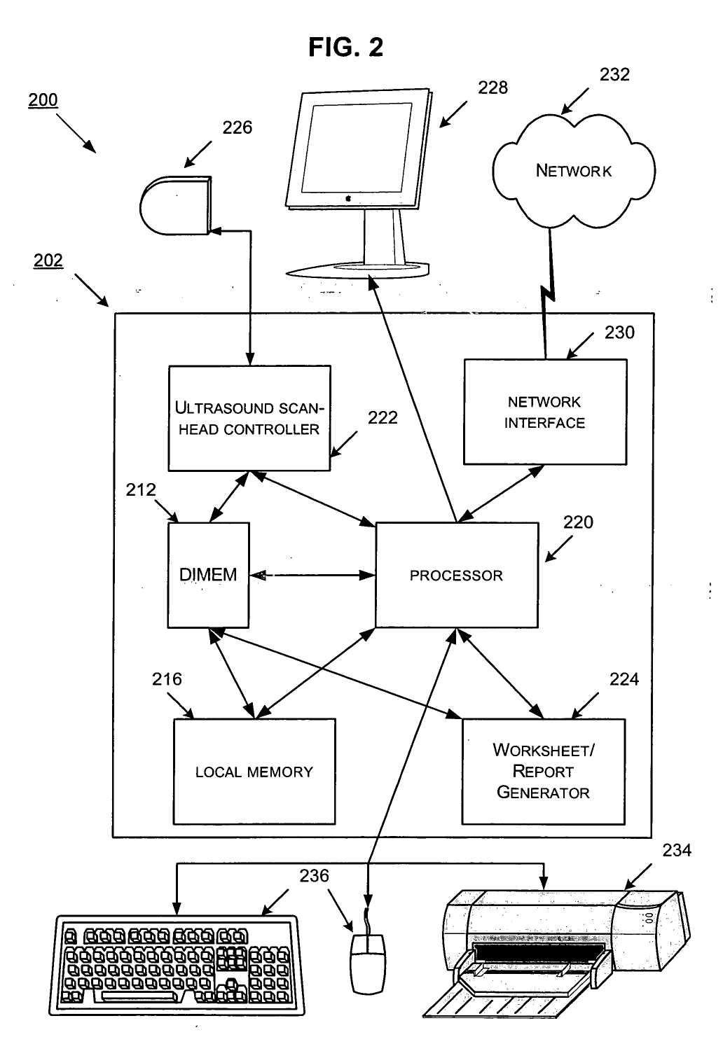

An ultrasound system in which DIMEM can be embodied is illustrated in FIG. 2. What is shown is one ultrasound station 202, although it can be part of a networked system that seamlessly integrates information throughout the enterprise. Such system would require each ultrasound station to include a network interface 230 for communicating through the network 232. The networked system can be configured, for example, as a hospital information system with distributed processing and storage capacity and including one or more servers and non-volatile storage devices such as CD-ROMs and/or redundant array of inexpensive disks (RAID). Under control of the processor 220, ultrasound exam data can be stored in local memory 216 or RAID. In ultrasound systems for medical diagnosis, the worksheet can be exported to networked DICOM servers (DICOM stands for Digital Imaging and Communications in Medicine).

In this system, a report generator 224 populates the worksheets and reports with scan results obtained via the scan head 226 under the control of the ultrasound scan-head controller 222 and with further processing control by the processor 220. The DIMEM feature is shown as a separate element of the system because it is functionally a completely unique add on to this system. However, structurally, the DIMEM feature can be embodied in various portions of the system's hardware and software modules. To that end, the system may have one or more types of command input devices 236 for enabling the DIMEM feature, for selecting a measured value in relation to which the DIMEM feature is invoked, for calling out one of the DIMEM menus, and for selecting a menu item. These input devices can be any combination of input facilities such as keyboard, dedicated hard or soft keys, touch screen, track ball, mouse, stylus, voice command recognition, and the like. The printer 234 as well as the monitor 228 are output devices used for documenting and showing the scan results, respectively.

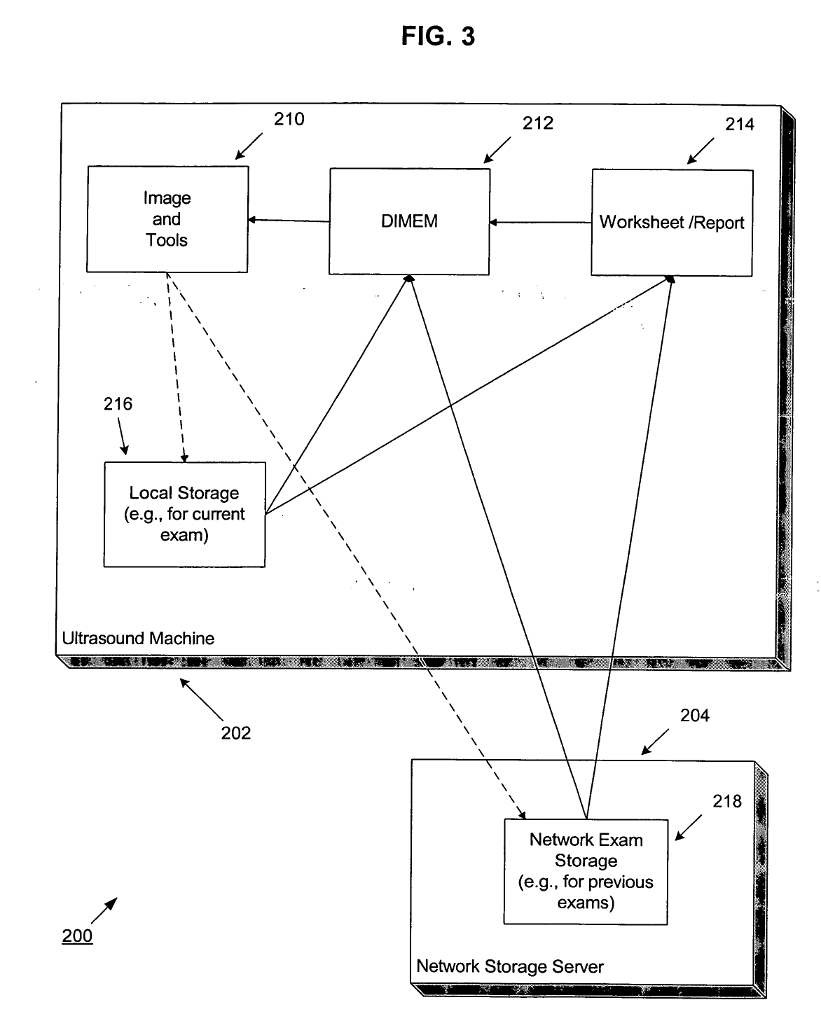

FIG. 3 is a diagram of the relationship between functional components of an ultrasound system 202 with the DIMEM feature 212. As shown in this example, scan results 210 for the current exam are stored in local storage (local memory) 216 and for the previous exams the scan results are stored, via the network, in a storage server 204 (with RAID facilities 218). For exams where DIMEM 212 is enabled, the scan results along with the tool identification and state at time of measurement are preserved. When an image is recalled from the worksheet 214, DIMEM 212 facilitates restoration of the tool and the state it had at time of measurement along with the recalled image. For previous exams, the tools and their state are downloaded from the storage server 204 to the local memory 216 for display, further review and manipulation. Moreover, scan results 210 from the current exam can be swapped with those of a previous exam, or they can remain in the local storage 216 while scan results from the previous exam are downloaded from the server 204. Again, with DIMEM invocation, the displayed images remain frozen but the tools are active and adjustable. The dashed lines represent storage of modified measurement values.

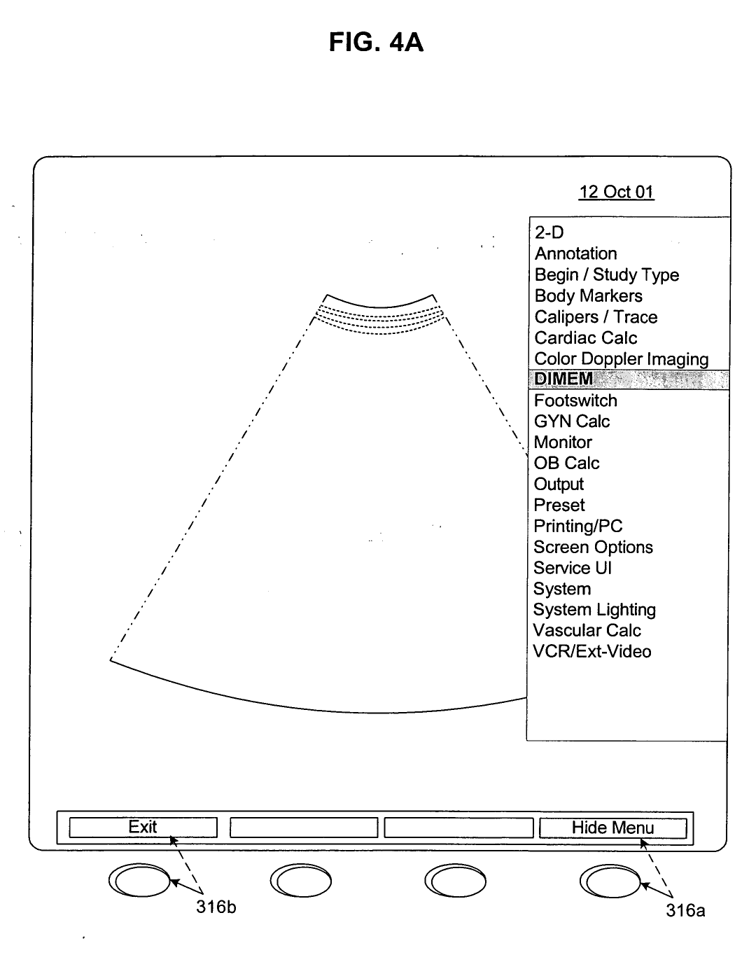

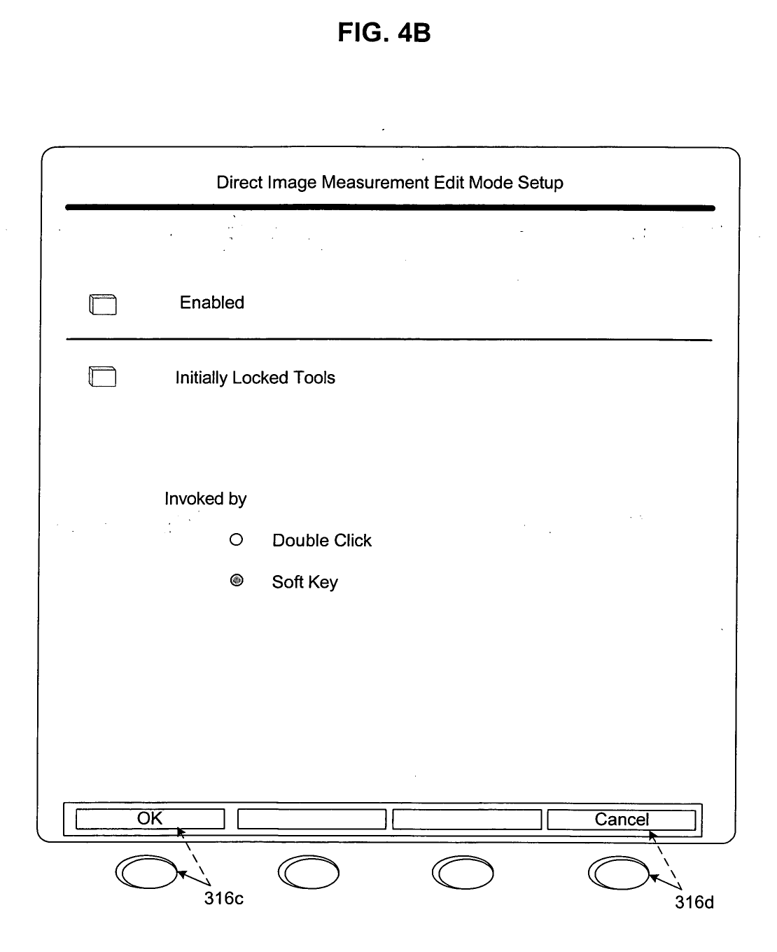

The DIMEM feature can be enabled in a number of ways. FIG. 4A provides the pre-exam setup screen with an exam menu that includes DIMEM and ultrasound scan choices. It indicates one way for enabling the DIMEM feature which is through menu selection and then through DIMEM setup. FIG. 4B illustrates the DIMEM setup screen which is invoked when the DIMEM feature is selected in the exam menu. In both FIG. 4A and FIG. 4B, the soft keys 316a-d allow navigation in and out of the screens as well as accepting and canceling the DIMEM setup and hiding the menu. When the DIMEM setup is accepted, in this example via the soft key labeled “OK” 316c, the pre-exam screen returns to the display. The hide menu function, invoked via the soft key labeled “Hide Menu” 316a, removes the ultrasound scan selections menu from the screen and allows the scan to proceed on command. The reverse of this function would be the show menu function and it would be invoked with a “Show Menu” soft key (not shown here). This function would be the option when the program is invoked and the pre-exam setup screen shows up for the first time or when the menu has been hidden. When the menu is shown the cursor can navigate up-down the menu to select an item, such as “OB Calc.” Once the item is selected the menu can be hidden and the exam can proceed.

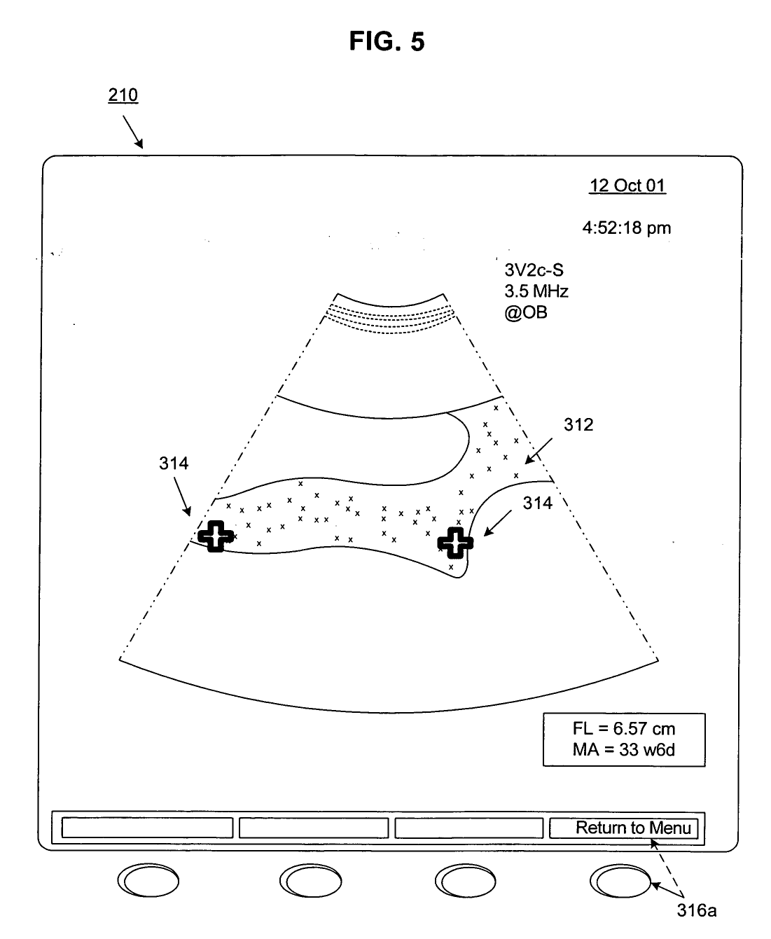

With the DIMEM feature enabled, the scan proceeds normally except that the scan results are saved along with the tool information, including its state at time of measurement. What this would look like on the screen is an image of the scanned area with whatever objects are found and the tool elements that are used for taking the measurement. FIG. 5 shows one freeze frame example of an ultrasound scan with the distance caliper set for length measurement. In this example, the scan results 210 include the image of a femur 312 and the two reference points 314 of the distance caliper set for femur length measurement. Then, if the DIMEM feature has been enabled for the exam, the measurement value is saved along with the image identification (ID), tool ID, tool state at time of measurement, exam ID and possibly also patient ID. As will be later explained in more detail, the DIMEM feature enables recall of this image along with the measurement tool which is restored to its original state at time of taking the measurement.

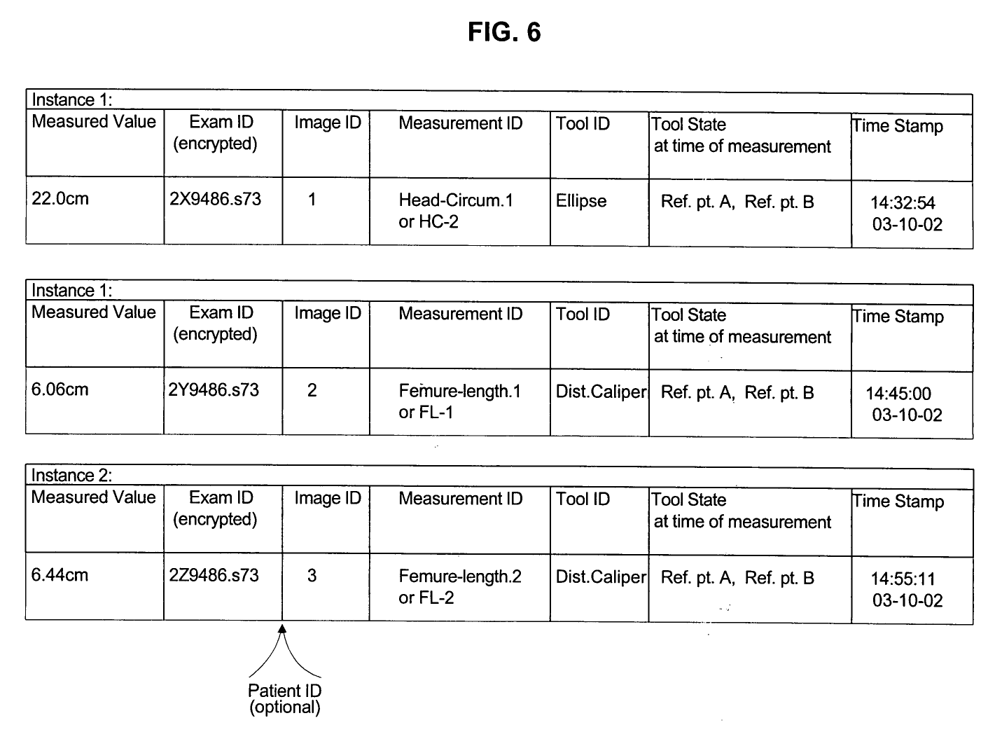

As mentioned, each measured value is an instance. Suppose that for a particular image there are three measurements taken. The three measurements produce three instances of measurement value, two instances of femur length measurement value and one instance of circumference measurement value. FIG. 6 shows the data saved with the image, and we assume that in this implementation each instance has a separate record. As shown, each records includes a number of fields the more important of which are the measured value, image ID, tool ID, tool settings at time of measurement, and exam ID and measurement ID. The exam ID is preferably presented and saved in encrypted form to preserve privacy and limit access. The image ID can be simply a numeric character or any other form of identification. The measurement ID indicates the type of measurement and instance number, particularly if more than one measurement of the same type is taken in this exam. For example, a measurement ID of “FL-2” stands for femur length second instance. A time stamp is typically added to the record to allow dating the scan results. The patient ID need not be but it can be included in the record in addition to the Exam ID.

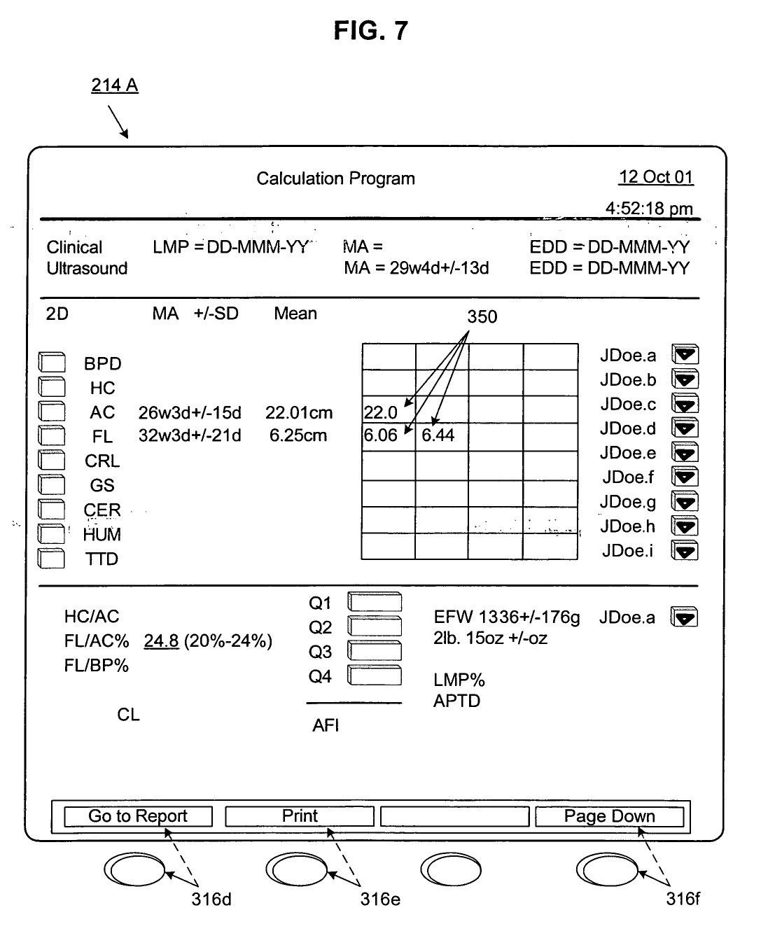

At the end of a scan the sonographer can bring up a worksheet that is populated with the scan results. FIG. 7 shows an example of a structured worksheet with tabulated measurement values 350. The instances of the measurement values 350 are tabulated based on the type of measurement, e.g., femur length (FL) or abdominal circumference (AC) and they are associated with a specific computed quantity derived from the measurement value (e.g., MA) and a computation algorithm (e.g., JDoe.c and JDoe.d). Each instance can be adjusted or refined in a number of ways. Suppose that the measured value 22.0 cm is of interest to the sonographer and he navigates the cursor to this instance. From this cursor location, namely, from this measured value, the sonographer can recall the image linked to it. If the DIMEM feature is enabled for the exam, DIMEM is invoked upon selecting the measured value by touching it, pressing a button or otherwise.

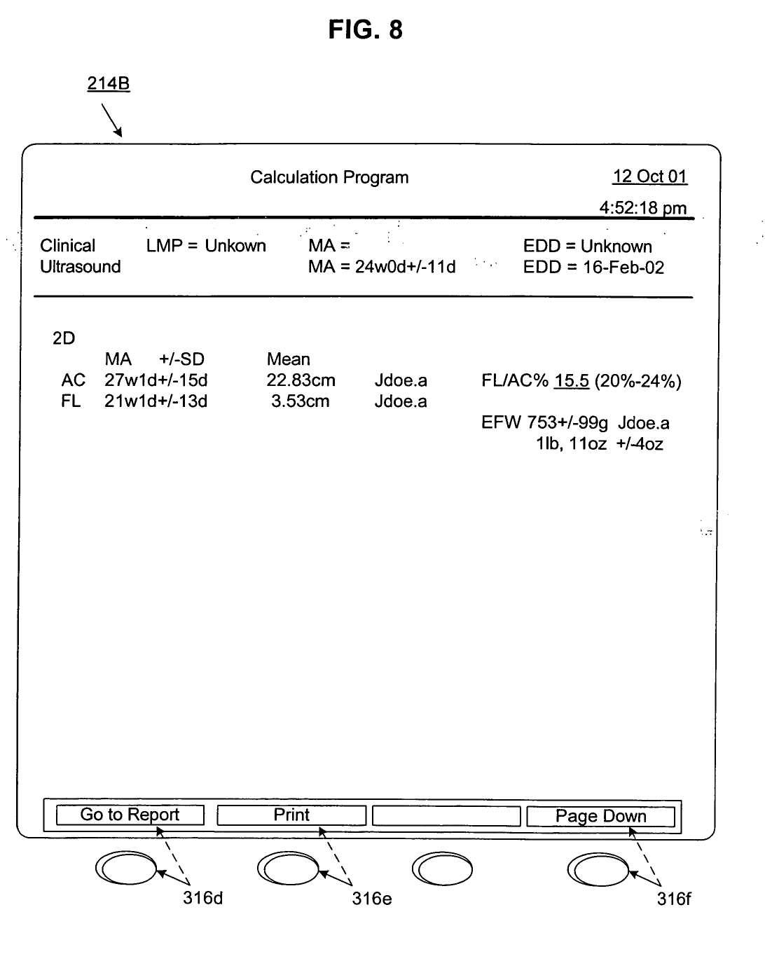

As further shown, there are soft keys 316d-f that operate to activate the print, report and page-down functions. The soft key labeled “Go to Report” activates the report function and causes the system to generate a structured report. FIG. 8 is an exemplary report derived from the worksheet. As can be seen, the report is constructed with data of interest relevant for the purpose of the particular ultrasound exam.

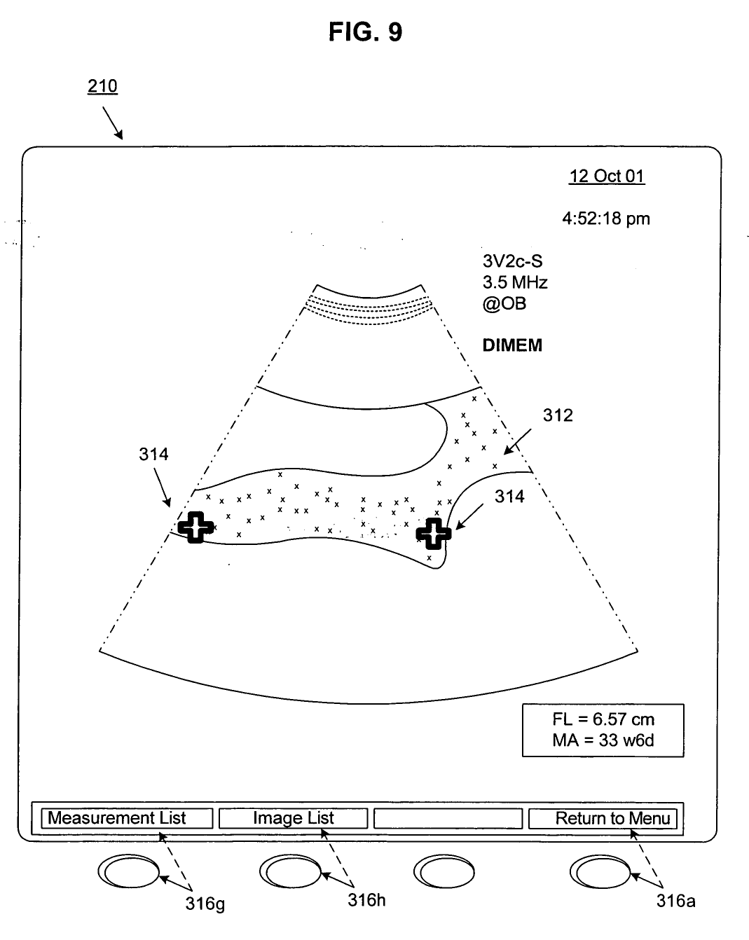

Returning for a moment to FIG. 7, we have noted above that DIMEM is invoked upon selecting the measured value, say 22.0 cm. From there, DIMEM links this measured value to the image identified with that measured value, as discussed above. The record for this instance includes, among other things, the exam ID and image ID which allow the system to locate the associated image in memory, local or remote. Once the image is located it is recalled and displayed. FIG. 9 illustrates the recalled scan results previously shown in FIG. 5. As can be seen, because DIMEM is invoked the recalled scan results include the tool restored to its original state at time of taking the measurement. The tool is shown superimposed on the freeze frame of the ultrasound scan with the detected image 312. Then, although in this case the image remains frozen, the restored tool is active and can be manipulated by moving the reference points 314 to adjust the measured value as needed. There may be cases where a measurement is taken on a sequence of recorded images, a “cine clip” as they are known. Or there may be new measurement types that does not require a frozen image.

Notably, with DIMEM activated this image can be a starting point to a series of linked images. One way DIMEM is designed to provide this capability is through measurement and image lists. Access to the measurement and image lists is provided via the soft keys labeled “measurement list” 316g and “Image List” 316h, respectively. The measurement list includes all the instances of the same kind of measured value taken in any of the exams involving the identified patient. In this example, the measurement list will include all the FL measurements taken in all the exams involving the patient identified with these ultrasound scan results 210. Once this list is presented on the screen, the sonographer can recall any of the images associated with the listed measurement values. This selection will link the newly selected measured value to its associated image, tool and tool state. The image associated to each instance can be the same image as before or a different image. The associated tool is not necessarily similar in all instances of the particular measured value because more than one measurement tool may be available for the measurement. However, for each instance, the tool is restored to its particular state at time of taking the particular measurement (and this state is obtained from the record).

From the screen of the newly recalled image, the measurement list can be invoked, another measured value can be selected to recall its associated image, tool and tool state, and so on. In other words, DIMEM enables the image lists to be recursive.

The same is true with the measurement lists which are also recursive. A measurement list includes all the measurement values, of all kinds, for a particular ultrasound scan involving the identified patient. Once a measurement list is invoked, the selected measurement value is selected to recall the image and, in particular, to restore the associated tool to its state at time of taking the measurement. Various types of measurements are taken with various kinds of appropriate tools, and for one exam there may be different tools restored for different kinds of measurements.

After an image is recalled and the measurement tool is manipulated to adjust the measurement a new measurement value is calculated. The new measurement results can be tabulated in a newly-generated worksheet and report. Additionally, measurement values tabulated in the new worksheet can be the starting point for an image recall once again as described above.

Note that both the image list and measurement lists are available with each recalled image. Thus, from each screen the sonographer can invoke either an image list or a measurement list and recall an image associated with one or the other. This flexibility allows a sonographer to review the entire exam history for the identified patient in any order seamlessly.

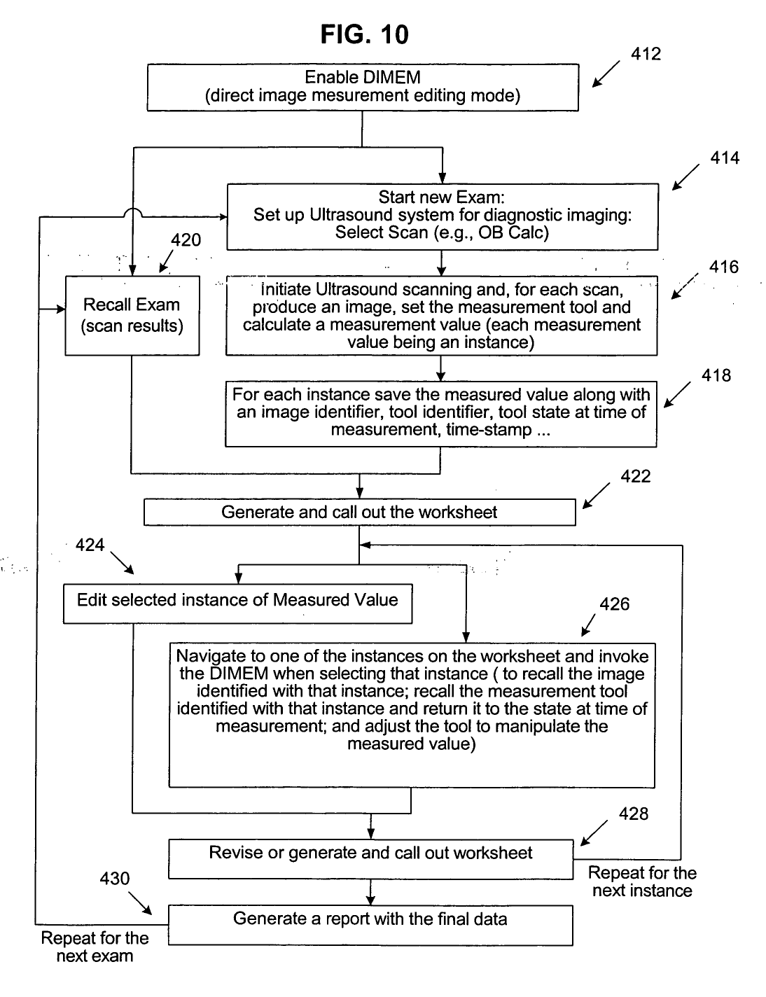

FIG. 10 provides an overview of the workflow with DIMEM enabled, starting with step 412. At this point the sonographer may choose to recall the ultrasound scan results of an exam taken earlier 420 or to start a new exam 414. In order to start a new exam, a pre-exam setup includes selecting the type of scan, e.g., OB Calc. For each ultrasound scan initiated thereafter, scan results are obtained including invoking a measurement tool and taking a measurement after setting the measurement tool 416. For each instance, the measured value is saved in memory 216, and, as indicated before, its associated record includes information such as the measurement ID, tool ID, tool setting, image ID and exam ID. Upon completing the exam, a worksheet is generated 422 to view the results in tabulated form. Incidentally, if, instead of a new exam, a previous exam is recalled 420 the worksheet for such exam is called out 422.

When the worksheet is made available, the measurement values are shown tabulated and each of them can be edited in place 424. That is, the sonographer can navigate the cursor to a particular instance of choice and use the keyboard (or other means) to edit the measurement value, say from 22.0 to 22.3. In this case, the worksheet is revised accordingly 428. Alternatively, the DIMEM feature can be invoked and, as previously explained, once the image and tools are restored, the measurement value can be adjusted by manipulating the measurement tool 426. This time, the worksheet is generated with the new measurement values 428.

From the worksheet, the next instance can be selected and either edited or adjusted after invoking DIMEM and recalling its associated image and tools, as explained above. The process can repeat as many times as needed to address all or fewer than all the instances tabulated in the worksheet. For each exam, once the worksheet is made available 428, the report can be generated to document the results 430. Then, if need be, the above-described process can repeat for a new exam or a recalled exam.

In sum, the DIMEM feature introduces flexibility into the ultrasound exam process and, as a result, it improved the workflow associated with it. This flexibility allows a sonographer to seamlessly review, in any order, the entire history of exams. This is useful in medical diagnosis as illustrated above and in other diagnostic imaging applications. And, while the present invention has been described in considerable detail with reference to certain versions thereof, other versions are possible. Therefore, the spirit and scope of the appended claims should not be limited to the description of the preferred versions contained herein.

What is claimed is:

1. An ultrasound system with direct image editing mode, comprising:
a worksheet-report generator with input and output, the input for receiving ultrasound scan results of an exam which include an image and one or more instances of a measurement value associated with the image and the output for providing a worksheet populated with instances of the ultrasound scan results;
one or more measurement tools with a setting for taking a measurement, wherein each instance of a measurement value is obtained with a measurement tool;
a storage; and
a direct image editing mode (DIMEM) module operatively connected to the worksheet-report generator and storage and configured to preserve in the storage each instance of each measurement value along with information on its associated tool and its setting so that when the image associated with such instance is recalled for further review the measurement tool used for obtaining such instance is restorable to its setting at the time of taking the measurement for allowing further adjustment of such instance.

2. An ultrasound system as in claim 1, further comprising:
a pointer navigable between the instances on the worksheet; and
a touch activated command input operatively associated with the pointer and the DIMEM module so that a command provided to the DIMEM from the touch activated command input is associated with the instance to which the pointer navigates.

3. An ultrasound system as in claim 1, wherein the DIMEM module includes a command identifier configured to receive and identify a DIMEM enable command, a DIMEM invoke command, a retrieve instance record command, a recall image command and a restore tool command.

4. An ultrasound system as in claim 1, wherein the measurement tool associated with an instance is restorable to an active mode and modifiable for adjusting such instance.

5. An ultrasound system as in claim 1, wherein the storage is any combination of one or more of local, removable and remote memory.

6. An ultrasound system as in claim 5, wherein the removable storage is a CD-ROM and the remote storage is RAID (redundant inexpensive array of disks), storage server, or both.

7. An ultrasound system as in claim 2, wherein the touch activated command input is any one of a button, a soft key, a stylus, voice activated input, and a touch screen.

8. An ultrasound system as in claim 1, wherein the storage includes sufficient space for holding an exam history with the ultrasound scan results of one or more exams, wherein the DIMEM module enables seamless review of the exam history in any order.

9. An ultrasound system as in claim 5, further comprising a network wherein the remote storage is accessible via the network.

10. An ultrasound system as in claim 1, further comprising:
a processor; and
an ultrasound scan-head controller for providing the ultrasound scan results to the worksheet-report generator under supervision of the processor.

11. An ultrasound system as in claim 9, further comprising:
a processor; and
a network interface operatively interposed between the network and the processor.

12. An ultrasound system as in claim 1, further comprising a display, a printer, or both.

13. An ultrasound system as in claim 1, wherein the worksheet-report generator is configured for generating a report with the ultrasound scan results that populate the worksheet.

14. An ultrasound system as in claim 1, wherein the DIMEM module includes a touch activated command input for image list invocation and measurement list invocation, and wherein the storage is accessible to the DIMEM module for obtaining therefrom an image list and a measurement list, the image and measurement lists being obtainable from any freeze frame containing a recalled image.

15. An ultrasound system as in claim 1, wherein the storage is configured with space for holding one record for each instance, each record including an image identification (ID) an exam ID, a measurement ID, a tool ID, the setting of the tool at time of measurement, and a time stamp.

16. An ultrasound system as in claim 1, wherein the instances associated with the ultrasound scan results are tabulated on the worksheet.

17. A method for improving ultrasound workflow with direct image measurement editing mode, comprising:
enabling the direct image measurement editing mode (DIMEM) in an ultrasound system;
obtaining ultrasound scan results for an exam which include an image and one or more instances of a measurement value associated with the image, wherein each instance of a measurement value is taken by a measurement tool with settings for taking a measurement;
saving the ultrasound scan results along with information about the measurement tool and its settings at time of measurement;
providing a worksheet populated with the instances;
navigating to one of the instances on the worksheet which is of interest;
invoking the DIMEM, and, with the invoked DIMEM, recalling the image associated with the instance of interest and restoring the tool associated with the instance to its settings at time of the measurement;
reviewing the instance of interest, adjusting the instance of interest, or both; and
generating a new worksheet with the instance of interest, whereby the recall of the image along with restoration of the associated tool to its settings at time of measurement improves the workflow.

18. A method as in claim 17, wherein the exam is new so that before it begins the method further comprises the step of setting the ultrasound system for the new exam.

19. A method as in claim 17, wherein the exam was previously conducted and its ultrasound scan results were stored in memory such that to obtain the scan results for this exam it is recalled from memory.

20. A method as in claim 17, wherein each instance is editable directly on the worksheet before, after or instead of invoking the DIMEM.

21. A method as in claim 17, further comprising generating a report from the worksheet.

22. A method as in claim 17, wherein the steps of obtaining, providing, navigating, invoking, reviewing and generating are repeated for another exam.

23. A method as in claim 17, wherein the steps of navigating, invoking, reviewing and generating are repeated for another instance.

24. A method as in claim 17, wherein invoking the DIMEM allows invocation of a measurement list and an image list from each recalled image, and wherein the measurement and image lists are recursive to allow seamless navigation through exams in any desired order.
